(19) United States
(12) Patent Application Publication
(10) Pub. No.: US 2013/0023590 A1
(43) Pub. Date: Jan. 24, 2013

(54) AQUEOUS COMPOSITIONS COMPRISING RESVERATROL AND METHODS FOR MAKING THEM

(71) Applicant: Cezar Constantin Giosan, Jackson Heights, NY (US)

(72) Inventor: Cezar Constantin Giosan, Jackson Heights, NY (US)

(21) Appl. No.: 13/187,753

(22) Filed: Jul. 21, 2011

(51) Int. Cl. A61K 31/05; A23L 1/305; A23L 2/02; A23L 1/303; A23L 1/304; A23F 3/00; A23L 1/30; A23L 1/302

(52) U.S. Cl. 514/733; 426/648; 426/72; 426/73; 426/74; 426/597; 426/599; 426/60

(57) ABSTRACT

A method is provided for solubilizing and stabilizing resveratrol in aqueous solutions free of solvents other than water. Resveratrol is solubilized and stabilized by forming complex structures with positively-charged substances in solution, such as caffeine, vitamins, amino acids and/or metallic ions. Additionally, these solutions can be further enhanced by adding any combination of health-promoting compounds, such as teas, plants and fruits, juices, protein or fiber. Aqueous solutions prepared by the method are also disclosed.

### BACKGROUND OF THE INVENTION

1. Field of Invention

This invention relates to compositions comprising resveratrol and more particularly to aqueous solutions containing resveratrol and methods for making them.

2. Description of Related Art

Resveratrol (3,4′,5-trihydroxystilbene) has been found in numerous in vitro and in vivo studies to have a wide range of beneficial health effects.

Resveratrol is a powerful anti-oxidant (See, e.g., Frankel et al., Lancet, 341(8852):1103-1104, 1993; Stojanovic et al., Archives of Biochemistry and Biophysics 391(1): 79-80, 2001).

Resveratrol has anti-inflammatory properties. It inhibits the activity of several enzymes in vitro, including cyclooxygenase and lipoxygenase (Donnelly et al., Am. J. Physiology Lung Cellular and Molecular Physiology 287(4):L774-783, 2004), and may inhibit pro-inflammatory transcription factors, such NFKB or AP-I (de la Lastra et al., Molecular Nutrition & Food Research, 49(5):405-430, 2005).

Resveratrol has anti-clotting properties. Resveratrol has been found to inhibit platelet aggregation in vitro (Kirk et al., Blood Cells Molecules and Diseases, 26(2):144-150, 2000). Platelet aggregation is one of the first steps in the formation of a blood clot that can occlude a coronary or cerebral artery, resulting in a myocardial infarction or a stroke, respectively.

Resveratrol promotes longevity. In yeast, caloric restriction stimulates the activity of an enzyme known as Sirtuin 2. Providing resveratrol to yeast increased Sirtuin 2 activity in the absence of caloric restriction and extended the replicative lifespan of yeast by 70%. Caloric restriction is also known to extend the lifespan of a number of species, including mammals (Baur et al., Nature, 444(7117):337, 2006).

Resveratrol has anticarcinogenic activity. In vitro research has shown that resveratrol inhibited the effects of breast, skin, gastric, colon, esophageal, prostate and pancreatic cancer cells, as well as leukemia (Sinclair et al., Nature Review Drug Discovery 5(6): 493-506, 2006). Resveratrol has been found to inhibit proliferation and induce apoptosis in a number of cell lines (Fulda et al: Cancer Detection and Prevention, 30(3): 217-223, 2006). Resveratrol increases vitamin 3's steroid mediated inhibition of breast cancer (Lu et al: J. of Cellular Physiology, 179: 297-304, 1999).

The effect of resveratrol on the biotransformation of enzymes in another mechanism by which resveratrol is anticarcinogenic. By inhibiting the expression and activity of certain cytochrome P4 50 enzymes, trans-resveratrol may help to prevent cancer by decreasing exposure to these activated carcinogens (Chen et al., Carcinogenesis 25(10):2005-2013, 2004).

Certain dietary supplements on the market contain resveratrol powder in different doses. Research has shown, however, that, although highly absorbed, the bioavailability of oral resveratrol in humans is very low (Walle et al., Drug Metabolism and Disposition, Vol. 32 (12) 1377-1382).

Resveratrol belongs to a class of polyphenolic compounds called stilbenes. It is fat-soluble compound that occurs in trans and cis configurations. Trans-resveratrol has been shown to be the more active configuration with regard to health benefits.

Trans-resveratrol is usually supplied as crystalline powder. A stock solution may be made by dissolving the resveratrol in an organic solvent such as ethanol, dimethyl sulfoxide or dimethyl formamide. The solubility of trans-resveratrol in these solvents is approximately 65 mg/ml. These solutions can be further diluted partially in water, but the product will contain organic solvents. Red wine (which contains about 10%-15% ethanol) contains 0.4 mg/liter-13 mg/liter resveratrol.

The poor solubility of resveratrol in water has limited its use as a nutritional supplement. According to Product Information Sheet RS010 of Sigma-Aldrich Co. (1997), resveratrol is soluble in water (with heating) at 3 mg/100 ml (i.e., 30 ppm).

Despite the poor solubility of resveratrol in water, it is desired to provide a nutritional product comprising an aqueous composition of resveratrol. It is further desired to provide a nutritional product comprising an aqueous solution of resveratrol and other nutritional supplements, such as vitamins, amino acids and minerals.

All references cited herein are incorporated herein by reference in their entireties.

### BRIEF SUMMARY OF THE INVENTION

Accordingly, a first aspect of the invention is an aqueous solution comprising: (a) resveratrol; (b) at least one first positively-charged substance selected from the group consisting of caffeine and an amino acid; and (c) water, wherein the solution is substantially free of solvents other than water and is sufficiently stable such that greater than 30 ppm of the resveratrol remains in solution after 30 days storage at 25° C.

A second aspect of the invention is a method for providing the aqueous solution of the invention, said method comprising the steps of: (a) mixing resveratrol, the first positively-charged substance and water at a solubilizing temperature to provide a heated solution; and (b) cooling the heated solution to provide the aqueous solution.

### DETAILED DESCRIPTION OF PREFERRED EMBODIMENTS OF THE INVENTION

The invention makes use of the enhanced solubility of resveratrol in water at elevated temperatures to provide a heated solution having a high concentration of resveratrol, and using stabilizing agents in the heated solution to maintain the concentration of the resveratrol in an aqueous solution (substantially free of solvents other than water) above 30 ppm after cooling to room temperature (i.e., about 25° C.). It has been discovered that resveratrol molecules, which are negatively charged, can be bound to positively-charged molecules to form stable complexes in aqueous solution.

Compositions of the Invention

The invention provides aqueous solutions of resveratrol stabilized with at least one first positively-charged substance selected from the group consisting of caffeine and an amino acid and optionally at least one second positively-charged substance selected from the group consisting of a vitamin and a metal ion, wherein the solution is substantially free of solvents other than water and is sufficiently stable such that greater than 30 ppm of the resveratrol remains in solution after 30 days storage at 25° C.

Resveratrol is readily available for purchase from commercial suppliers, such as Sigma-Aldrich (e.g., trans-resveratrol, Product No. R5010). Resveratrol can be chemically synthesized (see, e.g., Farina et al., “An improved synthesis of resveratrol.” Nat. Prod. Res. 20 (3): 247-52 (2006)) or isolated from certain plants and legumes, including Japanese knotweed (see, e.g., Powell et al., Phytochemistry 35, p. 335, 1994), wine and grapes (see, e.g., Goldberg et al.; J. Agric. Food Chem., 43, p. 1820, 1995 and Cellotti et al., “Resveratrol content of some wines obtained from dried Valpolicella grapes: Recioto and Amarone., J chromatogr A (Netherlands) 730: 47-52, 1996), and from peanut plant cultures (Kindl et al., U.S. Pat. No. 5,391,724). The resveratrol is preferably in the trans configuration.

The concentration of resveratrol in solution is dictated more by the purpose of the solution than it is by solubility concerns. Accordingly, resveratrol is preferably provided in solution in an amount effective to achieve at least one desired biological effect (i.e., a “biologically effective amount”), such as those described above. Thus, for example, a single 12 fluid ounce (355 ml) serving of liquid nutritional supplement can contain 10 mg or 25 mg or 50 mg or 100 mg or 250 mg or 500 mg of resveratrol.

The concentration of resveratrol in solution is preferably in excess of the maximum concentration of resveratrol in solution at 25° C. absent the positively-charged substances. Thus, the concentration at 25° C. is preferably: >30 ppm or ≧40 ppm or ≧50 ppm or ≧100 ppm or ≧500 ppm or ≧1000 ppm or ≧10000 or ≧20000 ppm. In certain embodiments, the concentration of resveratrol in solution is from 30 ppm to 10000 ppm or from 40 ppm to 1000 ppm or from 40 ppm to 7000 ppm.

It is preferred that the aqueous solution be sufficiently stable such that the concentration of resveratrol in solution at 25° C. is preferably greater than 30 ppm after 30 days storage at 25° C., and more preferably after 90 days at 25° C., and still more preferably after six months at 25° C. It is particularly preferred that the aqueous solution be sufficiently stable such that the concentration of resveratrol in solution at 25° C. on day 30 is at least 95% or 90% or 75% or 50% of the concentration of resveratrol in solution at 25° C. at the beginning of the stability test (i.e., on Day 0). It is more particularly preferred that the aqueous solution be sufficiently stable such that the concentration of resveratrol in solution at 25° C. on day 90 is at least 95% or 90% or 75% or 50% of the concentration of resveratrol in solution at 25° C. on Day 0.

The resveratrol is stabilized in solution by positively-charged substances capable of forming stable complexes with resveratrol, which is negatively-charged. A positively-charged substance as defined herein is a cation, a molecule having a net positive charge, or a molecule having a localized positive charge that is available for bonding to resveratrol. Examples of positively-charged substances suitable for use in the invention include, but are not limited to: (a) positively-charged amino acids, including Lysine, Histidine, Arginine; (b) positively-charged vitamins; (c) positively-charged metal ions, including ions of Ca, Na, K and Mg; and (d) other positively-charged substances, including caffeine.

The positively-charged substances are preferably present in the aqueous solution in an amount effective to maintain substantially all of the resveratrol in solution at 25° C. In certain embodiments, the positively-charged substances are present in biologically effective amounts.

The positively-charged substances enable the resveratrol to remain in an unheated solution substantially free of solvents other than water. The expression “substantially free of solvents other than water” as used herein means that the aqueous solution contains no detectable amounts of solvents other than water, or contains such other solvents in collective amounts insufficient to raise the solubility of resveratrol above 40 ppm at 25° C. (in an aqueous solution free of the positively-charged substances). In certain embodiments, the aqueous solution contains solvents other than water in volume percentages less than 5% or 4% or 3% or 2% or 1% or 0.1% or 0.01%.

In addition to resveratrol, water and positively-charged substances, the aqueous solution can optionally include additional ingredients suitable for consumption (e.g., as in a beverage) and/or administration (e.g., as in a pharmaceutical dosage form) to humans or other organisms. Non-limiting examples of these additional ingredients are vitamins, amino acids, tea, protein, fiber, plant extracts, vegetable juice and fruit juice.

In the case of ingredients (including positively-charged substances) for which a Recommended Daily Allowance (RDA) has been established in the United States, the ingredients are preferably provided in a single serving or dosage form in an amount of 1% to 500% of the RDA. In certain embodiments, mineral ingredients are provided in an amount of at least 10%, more typically 50% to 300%, of the RDA per serving or dosage form. In certain embodiments, vitamins are provided in an amount of more than 10%, preferably about 20% to about 100% and, most preferably, from about 50% to about 150% of the RDA. Of course, the preferred daily intake of any mineral or vitamin may vary with the user, and the compositions of the present invention may be adjusted accordingly.

Methods of the Invention

The preferred method for providing the aqueous solutions of the invention comprises three phases:

Solubilization and Stabilization of Resveratrol.

This initial phase consists of mixing resveratrol extract in water (e.g., DI-water, tap water, mineral water, or any other kind of potable water) at elevated temperatures for a specific period of time, until solubilization is achieved. This is followed by adding positively-charged molecules, such as caffeine, L-Lysine, L-Histidine, or their combinations, to the solution. The solution is then cooled to room temperature. This leads to the stabilization of resveratrol through the formation of hydrogen, van der Waals, or ionic bonds with the positively-charged molecules. To increase the quantity of resveratrol solubilized in water, an emulsifying agent suitable for use in foods and/or pharmaecuticals, such as polysorbate 80, can be mixed with water before adding the resveratrol extract. This aqueous solution will henceforth be referred to as “Primary Resveratrol Water” or PRW.

Enhancing the Activity of PRW with Amino Acids and Vitamins.

To obtain this solution (henceforth referred to as “Enhanced Resveratrol Water”, or ERW), four different methods can be employed:

a) Mixing PRW with various concentrations of lysate (sonicated, thermolyzed, etc.) Brewer's yeast at room temperature. Lysate yeast contains all water soluble vitamins and essential and non-essential amino acids. Through mixing, the resveratrol/caffeine/Lysine/Histidine structures will, based on their electrical polarities, further bind with other amino acids and vitamins in stable complex structures.

b) Mixing PRW with Brewer's yeast at elevated temperatures. This process lyses the yeast membranes, thus releasing the cells' contents (amino acids and vitamins). Through mixing, the resveratrol/caffeine/Lysine/Histidine structures will, based on their electrical polarities, further bind with other amino acids and vitamins in stable complex structures. The temperature is then lowered to room temperature and a sterile filtration is performed.

c) Mixing PRW with water-insoluble vitamins (A, E) and Brewer's yeast at elevated temperatures. This process ruptures the yeast, thus releasing the cells' contents (amino acids and vitamins). At the same time, some of the water-insoluble vitamins became soluble based on the lower tension of water's surface. Through mixing, the resveratrol/caffeine/Lysine/Histidine structures will, based on their electrical polarities, further bind with other amino acids and vitamins in stable complex structures. The temperature is then lowered to room temperature and a sterile filtration is performed.

d) Mixing PRW with any other available water-soluble vitamins (C and B complex) and amino acids such as Lysine, Histidine, L-Arginine, etc., at room temperature, for a specific period of time, to allow the solubilization process to take place. Through mixing, resveratrol/caffeine/Lysine/Histidine structures will, based on their electrical polarities, further bind with other amino acids and vitamins in stable complex structures.

Fortifying the Solution with Mineral Ions:

PRW or ERW are mixed at room temperature with various concentrations of salts containing mineral ions (Ca, Na, K, Mg). Through mixing, mineral ions will, based on their electrical polarities, bind to the resveratrol structures found in PRW or ERW, thus forming stable complex structures. This aqueous solution will henceforth be referred to as “Fortified Resveratrol Water”, or FRW.

Mixing any of the above-mentioned products (PRW, ERW, or FRW) with natural compounds, (e.g., plant extracts, plant compounds) or synthetic compounds with biological activity (e.g., synthetic coenzyme Q10, synthetic vitamins, etc.) at elevated temperatures and for a sufficient period of time for an infusion process to take place, followed by filtration.

Mixing any of the above-mentioned products (PRW, ERW, or FRW) with natural (e.g., plant extracts) or synthetic compounds having biological activity at elevated temperatures and for a sufficient period of time for an extraction process to take place, followed by filtration.

Mixing any of the above-mentioned products (PRW, ERW, or FRW) with concentrated fruits and/or vegetable pulps. This is a dilution process in PRW, ERW, or FRW.

Mixing any of the above-mentioned products (PRW, ERW, or FRW) with protein (whey, egg whites, etc.). This is a dilution in PRW, ERW, or FRW.

Mixing any of the above-mentioned products (PRW, ERW, or FRW) with fiber. This is a dilution in PRW, ERW, or FRW.

The invention will be illustrated in more detail with reference to the following Examples, but it should be understood that the present invention is not deemed to be limited thereto.

### EXAMPLES

Exemplary Procedures

A) Production of a solution containing solubilized resveratrol molecules stabilized in complex structures with caffeine, amino acids, vitamins, or metallic ions, in the absence of alcohol or any organic solvent. The method comprises three steps:

Solubilization and stabilization of resveratrol for the production of PRW.

Resveratrol extract powder (98% purity in concentrations of 0.001% to 0.2%) is added to deionized water, or any kind of potable water, with pH between 5.1 and 6.0 and stirred at a rate about 200 rpm-700 rpm. The system temperature is then increased to 65°-100° C. for 15-90 minutes, when a complete solubilization of resveratrol is achieved. Solubilization takes place based on a synergistic effect between heating and water's surface tension at these solubilizing temperatures. For example, water's surface tension in this range of temperatures (65°-100° C.) varies between 58.91 mJ/m² and 65.36 mJ/m² and increases to 72 mJ/m² at 25° C. At this value, resveratrol will start to precipitate from this solution (henceforth referred to as “Control”). To avoid this, positively-charged molecules of caffeine in concentrations of 0.005% to 2% are added to this aqueous solution under stirring. This will make resveratrol molecules bind with caffeine, thus stabilizing them in stable complex structures. Other positively-charged substances, such as L-Lysine, L-Histidine, or their combinations, or their combinations with caffeine, may also be used for resveratrol stabilization. The entire system is then cooled down to room temperature (25° C.). The resulting solution has a pH value between 5.1 and 7.2 and a conductivity value between 4 μS/cm and 120 μS/cm.

To increase the amount of resveratrol molecules solubilized in water, polysorbate 80, a food emulsifier, in concentrations between 0.01% and 10% can be mixed with water for a greater reduction of water's surface tension. After this is completed, resveratrol extract powder (98% purity, concentrations from 0.005% to 1%, is added to this aqueous solution and stirred at a rate of about 200 rpm-700 rpm. The system temperature is then increased to 40°-100° C. for 15-90 minutes, at which point a complete solubilization of resveratrol takes place. Because of polysorbate 80, the water's surface tension is low enough so that the resveratrol solubilized in water does not precipitate at room temperature. However, without the addition of positively-charged substances to stabilize the resveratrol, the water will change its color over time because of the oxidation processes of the solubilized resveratrol, the coloration process being amplified by the light. In order to avoid this, positively-charged molecules of caffeine in 0.005% to 5% concentration are added to this aqueous solution under stirring. The positively-charged caffeine binds to the resveratrol molecules, thus stabilizing them. Other positively-charged substances, such as L-Lysine, L-Histidine, or their combinations, or their combinations with caffeine, may be also used for resveratrol stabilization. Also, other water-soluble food emulsifiers, such as tri-potassium phosphate, may be used for resveratrol solubilization. The entire system is then cooled down to room temperature (25° C.). The resulting product has a pH between 5.1 and 7.2 and conductivity between 4.0 gS/cm and 180 gS/cm.

Enhancing the Activity of PRW with Amino Acids and Vitamins to Create ERW.

This can be achieved using the following methods, which result in a solution with a pH value between 3.1 and 7.2 and conductivity between 4 μS/cm and 300 μS/cm:

Mixing PRW with 0.05% to 35% sonicated or thermolyzed concentrated Brewer's yeast extract for 15 to 30 minutes, at a stirring rate between 50 rpm and 700 rpm. Through mixing, resveratrol/caffeine/Lysine/Histidine structures will bind in stable complex structures with other amino acids and vitamins from the yeast extract. The resulting product has a pH value in the range of 5.1 and 7.2 and a conductivity value in the range of 4 μS/cm to 90 μS/cm.

Mixing PRW with 0.001% to 1.0% dry Brewer's yeast and stirred at a rate between 50 rpm and 700 rpm. The temperature is briefly raised to 90°-95° C. for a period of about 10-20 minutes, which ruptures the yeast cells, releasing the cell content, such as amino acids and vitamins. The entire system is then cooled to room temperature and the mixing process continues for another 5 to 10 minutes. Through mixing, resveratrol/caffeine/Lysine/Histidine structures will bind further with other amino acids and vitamins, thus forming stable complex structures. The entire system is then filtered with 5 μm filter, followed by a 1 μm filter and, finally, a 0.22 μm sterile filter. Alternatively, yeast cells can be ruptured through any techniques used in yeast lysation.

Mixing PRW with 0.001% to 1.0% dry Brewer's yeast, 0.01% to 0.8% Vitamin A, 0.01% to 0.8% Vitamin E and stirred at a rate between 50 rpm and 700 rpm. The temperature is briefly raised to 90°-95° C. for about 10-20 minutes, which ruptures the yeast cells, releasing the cell content, such as amino acids and vitamins. In this process, as a result of the lower tension of the water's surface at elevated temperatures, some of the water-insoluble vitamins become soluble. The entire system is then cooled to room temperature and the mixing process continues for another 5 to 10 minutes. Through mixing, resveratrol/caffeine/Lysine/Histidine structures will bind further with other amino acids and vitamins, thus forming stable complex structures. The entire system is then filtered with 5 μm filter, followed by a 1 μm filter and, finally, a 0.22 μm sterile filter. Alternatively, yeast cells can be ruptured through any techniques used in yeast lysation.

Mixing PRW with any other water soluble amino acids and vitamins, such as Lysine, Histidine, L-Arginine, Glutamic acid, vitamin C, vitamin B complex, etc., in concentrations ranging from 0.001% to 1.0% at room temperature and stirred at 50 to 700 rpm for 10 to 30 minutes.

Production of FRW.

This can be accomplished via two methods:

PRW is mixed at room temperature with salts (0.01% to 1.5%) containing essential mineral ions of Ca, Na, K and Mg. Through the mixing process, minerals will bind to the structures of resveratrol/caffeine-Lysine/Histidine. The resulting solution has a pH value between 3.0 and 7.2 and conductivity between 4 μS/cm and 5000 μS/cm. This aqueous solution is henceforth referred to as “Fortified Resveratrol Water-M” (FRW-M).

ERW is mixed at room temperature with salts (0.01% to 1.5% concentrations) containing ionic Ca, Na, K and Mg. Through mixing, the mineral ions will bind to the structures of resveratrol/caffeine/amino acids/vitamins. The resulting solution has a pH value between 3.1 and 7.2 and conductivity between 4 μS/cm and 5000 μS/cm. This aqueous solution is referred to as Fortified Resveratrol Water-F (FRW-F).

B) Methods for producing solutions containing solubilized and stabilized resveratrol molecules in complex structures with caffeine, amino acids, vitamins, metallic ions, in the absence of alcohol or any organic solvent, supplemented with other health-promoting compounds such as teas, plants, fruits, protein, or fiber:

The production of these solutions can be realized via the following methods:

Infusion: 0.5% to 10% green tea powder extract is added to PRW, ERW or FRW at temperatures between 60° C.-100° C., with no agitation, for about 5-20 minutes. The resulting solution, which contains resveratrol and green tea, is then filtered to remove the plant, has a color specific to green tea, a pH between 2.5 and 7.1 and conductivity between 300 and 3000 μS/cm. Any plants and fruits extracts, such as strawberry, raspberry, chamomile, etc., can be used in this infusion process.

Extraction: 0.5% to 20% plants (e.g., dry fruits, vegetables, etc.) are added to PRW, ERW, or FRW at room temperature. The system is mixed for about 5 to 10 minutes and left for 4 to 48 hours for a complete extraction of the active ingredients to be achieved. The system is then filtered to remove the plants through successive size pores, up to 0.22μ. The resulting solution contains resveratrol, has a pH value between 2.3 and 7.5 and a conductivity of about 300 μS/cm to 10000 μS/cm. Any plants can be used in this extraction process.

Mixing 2% to 50% concentrated fruits and vegetable pulp, such as orange, tomato, etc. in PRW, ERW or FRW, for 5 to 30 minutes. The resulting beverage contains solubilized and stabilized resveratrol, has a pH value between 2.5 and 7.5 and conductivity between 300 μS/cm and 10000 μS/cm. Any concentrated frozen or non-frozen plants and fruits extracts can be used in this dilution process.

Mixing protein (powders or liquid, such as egg whites, whey, etc.) with PRW, ERW, or FRW for 5 to 30 minutes. The resulting solution contains solubilized resveratrol and has a pH value between 5.1 and 7.2. Any edible protein powders or liquids can be used in this dilution process.

Mixing fiber with PRW, ERW, or FRW for 5 to 30 minutes. The resulting solution contains solubilized resveratrol and has a pH value between 5.1 and 7.2. Any edible fiber can be used in this dilution process.

### Example 1

0.02% resveratrol powder, purity 98%, is mixed with deionized water at a stirring rate of about 300 rpm. The temperature is raised to 85° C. for 30 minutes. The system is then cooled down to room temperature (25° C.). The aqueous solution (referred henceforth to as “Control Water”) is then filtered through a sterile 0.22μ filter. The pH value is 5.80 and conductivity is about 3.9 μS/cm.

### Example 2

0.02% resveratrol powder, purity 98%, is mixed with deionized water at a stirring rate of about 300 rpm. The temperature is raised to 85° C. for 30 minutes. 0.2% caffeine powder is then added and the mixing process continues for an additional 5 minutes. The system is then cooled down under stirring to room temperature (25° C.). The resulting aqueous solution (referred henceforth to as “PRW-1”) is filtered through a 0.22μ filter. The pH value is 5.85 and conductivity is about 4.06 μS/cm.

### Example 3a

1.5% polysorbate 80 is mixed with deionized water at room temperature for 10 minutes. 0.1% resveratrol powder, purity 98%, is then added at a stirring rate of about 300 rpm. The temperature is then raised to 70° C. for 90 minutes. 1% caffeine powder is added and the mixing process is continued for another 15 minutes. The system is then cooled down to room temperature (25° C.). The resulting aqueous solution (referred henceforth to as “PRW-2”) is filtered through a sterile 0.22μ filter. The pH value is 5.53 and conductivity is about 23.6 μS/cm.

### Example 3b

10% polysorbate 80 is mixed with deionized water at room temperature for 10 minutes. 0.7% resveratrol powder, purity 98%, is then added at a stirring rate of about 300 rpm. The temperature is then raised to 50° C. for 90 minutes. 1% caffeine powder is added and the mixing process is continued for another 15 minutes. The system is then cooled down to room temperature (25° C.). The resulting aqueous solution has a concentration of resveratrol of 7000 ppm.

### Example 4a

### ERW-1

0.005% resveratrol powder, purity 98%, is mixed with deionized water at a stirring rate of about 300 rpm. The temperature is raised to 85° C. for 30 minutes. 0.2% caffeine powder is then added and the mixing process continues for another 5 minutes. 0.03% Dry Brewer's yeast is added and the temperature is raised to 90°-95° C. and kept there for 15 minutes. The system is then cooled down again to room temperature. The aqueous solution is filtered through a 5μ filter, followed by a 1μ filter and a sterile 0.22μ filter. The solution has a pH value of 6.05 and a conductivity of about 26.3 μS/cm.

### Example 4b

### ERW-2

0.03% Dry Brewer's yeast is added in PRW-1 (Example #2) and mixed at a stirring rate of about 300 rpm. The temperature is raised to 90°-95° C. and kept there for 15 minutes. The system is then cooled down again to room temperature. The aqueous solution is filtered through a 5μ filter, followed by a 1μ filter and a sterile 0.22μ filter. The solution has a pH value of 6.05 and a conductivity of about 26.3 μS/cm.

### Example 5

0.04% sea salts are added in PRW-1 water (Example #2) and mixed for 10 minutes at a stirring rate about 300 rpm. The aqueous solution (referred henceforth to as “FRW-M”) is filtered through 0.22μ. The pH value is 5.60 and conductivity is about 660 μS/cm.

### Example 6

### FRW

0.04% sea salt is mixed with ERW from example 4 for 5 minutes. The pH value is 5.75 and conductivity is about 530 μS/cm.

Analyses

1) Quantitative Determination of Resveratrol Content by HPLC

HPLC systems with two pumps, a gradient controller, an injector, C18 column 250×4.6 mm, UV detector, a data workstation, solvents: methanol, water and all standards can be obtained from Sigma. A well-mixed amount of sample is weighed into an extraction bottle. Add methanol and reflux the sample. Allow the sample to cool and filter it through a 0.45 um filter. If cleanup of the sample is needed use an SPE cartridge listed for extracting liquid samples.

If the sample has a high amount of resveratrol, dilute the sample with methanol and filter for HPLC analysis. If the sample has low amount of resveratrol, measure amount of the sample to be extracted and extract by SPE C18 cartridge. Concentrate and filter the sample for HPLC analysis.

Inject 20 μL of the standard and the test sample solutions into an HPLC with a gradient run of water and methanol at 1.0 mL per min and a temperature of 35° C. The mobile phase may be adjusted to ensure that system suitability passed. Resveratrol is detected with UV detector at 303 nm. Data are collected and analyzed by a data workstation using external standard calibration method. The following table shows the amount of resveratrol measured using the foregoing analytic protocol:

Trans-resveratrol amount in ppm

| | |
|---|---|
| After one month | After 3 months |

| Sample | Initial | 25° C. | 4° C. | 25° C. | 4° C. |
|---|---|---|---|---|---|
| Control | 31.64 | precip- | precip- | precip- | precip- |
| Water | | itated | itated | itated | itated |
| PRW-1 | 179 | Stable | Stable | Stable | Stable |
| PRW-2 | 1013 | Stable | Stable | Stable | Stable |
| ERW-1 | 46.52 | Stable | Stable | Stable | Stable |
| ERW-2 | 152 | Stable | Stable | Stable | Stable |
| FRW-M | 200 | Stable | Stable | Stable | Stable |

2) Quantitative Determination of Amino Acids.

Amino acids were measured with an HPLC Shimadzu LC 2010A with an AccQ Tag. The results were as follows:

| | Amino acid |
|---|---|
| Amino acid type | amount g/100 mL |
| Triptophan (essential) | 220 |
| L-Aspartic acid (non-essential) | 150 |
| L-Glutamic acid (non-essential) | 35 |
| L-Threonine (essential) | 20 |
| L-Histidine (non-essential) | 20 |
| L-Alanine (non-essential) | 155 |
| Valine (essential) | 60 |
| Lysine (essential) | 40 |
| Leucine (essential) | 90 |
| Isoleucine (essential) | 35 |
| L-Tyrosine (non-essential) | 10 |
| L-methionine (essential) | 5 |
| L-Phenylalanine (essential) | 10 |
| L-Proline (non-essential) | 10 |

### Example 7

### Resveratrol-Green Tea Beverage

Green tea (e.g., 1.5-gram bags) is added to PRW-1 from Example 2 and kept for 10 minutes at 90° C. Green tea is then removed. When the temperature reaches 25° C., the pH and conductivity are approximately 5.41 and 682 μS/cm, respectively.

### Example 8

### Resveratrol-Raspberry Tea Beverage

Raspberry Tea Powder (5 grams) is added to ERW from Example 4. The system is mixed for about 5 minutes at 100 rpm and left for a period of 48 hours for a complete extraction of the active ingredients to take place (water temperature about 25° C.). The raspberry tea powder is then removed through a 10μ filter. The pH and conductivity of this solution are 2.80 and 3200 μS/cm, respectively.

While the invention has been described in detail and with reference to specific examples thereof, it will be apparent to one skilled in the art that various changes and modifications can be made therein without departing from the spirit and scope thereof.

What is claimed is:

1. An aqueous solution comprising:
resveratrol;
at least one stabilizing agent; and
water,
wherein the solution is substantially free of solvents other than water and is sufficiently stable such that greater than 30 ppm of the resveratrol remains in solution after 30 days storage at 25° C.

2. The aqueous solution of claim 1, wherein the resveratrol is trans-resveratrol.

3. The aqueous solution of claim 1, wherein the stabilizing agent is a positively-charged substance selected from the group consisting of caffeine and an amino acid

4. The aqueous solution of claim 3, wherein the amino acid comprises at least one of Lysine, Histidine and Arginine.

5. The aqueous solution of claim 1, wherein the aqueous solution further comprises at least one of Vitamin A, Vitamin B, Vitamin C, Vitamin D, Vitamin E and Vitamin K.

6. The aqueous solution of claim 1, wherein the aqueous solution further comprises at least one cation of a metal selected from the group consisting of Ca, Na, K and Mg.

7. The aqueous solution of claim 1, wherein a concentration of the resveratrol in the solution is at least 40 ppm.

8. The aqueous solution of claim 1, further comprising an emulsifying agent.

9. The aqueous solution of claim 1, further comprising at least one ingredient selected from the group consisting of tea, protein, fiber, plant extracts, vegetable juice and fruit juice.

10. The aqueous solution of claim 3, further comprising at least one second positively-charged substance selected from the group consisting of a vitamin and a metal ion.

11. The aqueous solution of claim 10, wherein the resveratrol is trans-resveratrol at a concentration of 40 ppm to 7000 ppm, the amino acid comprises at least one of Lysine, Histidine and Arginine, the vitamin comprises at least one of Vitamin A, Vitamin B, Vitamin C and Vitamin E, and the metal ion comprises at least one cation selected from the group consisting of Ca, Na, K and Mg.

12. A method for providing the aqueous solution of claim 1, said method comprising the steps of:
mixing resveratrol, the stabilizing agent and water at a solubilizing temperature to provide a heated solution; and
cooling the heated solution to provide the aqueous solution.

13. The method of claim 12, wherein the resveratrol is mixed with the water prior to adding the stabilizing agent.

14. The method of claim 13, wherein the solubilizing temperature is 40° C.-100° C.

15. The method of claim 13, wherein the mixing step further comprises adding lysed Brewer's yeast to the water.

16. The method of claim 13, wherein the mixing step further comprises adding Brewer's yeast to the water at a temperature of 90° C.-95° C., and the method further comprises a step of performing a sterile filtration of the aqueous solution at room temperature.

17. The method of claim 15, wherein the mixing step further comprises adding water-insoluble vitamins to the water.

18. The method of claim 13, wherein the mixing step further comprises adding water-soluble vitamins and amino acids to the water.

19. The method of claim 13, wherein the mixing step further comprises adding to the water at least one salt of a metal selected from the group consisting of Ca, Na, K and Mg.

20. The method of claim 13, wherein the mixing step further comprises adding an emulsifying agent to the water at room temperature prior to adding the resveratrol.

21. The method of claim 13, wherein the mixing step further comprises adding to the water a second positively-charged substance selected from the group consisting of a vitamin and a metal ion.

22. The method of claim 13, wherein the method further comprises adding to the aqueous solution at least one ingredient selected from the group consisting of tea, protein, fiber, plant extracts, vegetable juice and fruit juice.

23. The method of claim 13, wherein a concentration of the resveratrol in the aqueous solution is at least 40 ppm.

24. The method of claim 13, wherein the resveratrol is trans-resveratrol at a concentration of 40 ppm to 7000 ppm, the amino acid comprises at least one of Lysine, Histidine and Arginine, the vitamin comprises at least one of Vitamin A, Vitamin B, Vitamin D, Vitamin E and Vitamin K, and the metal ion comprises at least one cation selected from the group consisting of Ca, Na, K and Mg.
